(19) United States
(12) Patent Application Publication
(10) Pub. No.: US 2002/0086067 A1
(43) Pub. Date: Jul. 4, 2002

(54) Catechins and green tea extract for the treatment of amyloidosis in alzheimer's disease and other amyloidoses

(76) Inventors: Paula Y. Choi, Bothell, WA (US); Gerardo Castillo, Seattle, WA (US); Alan D. Snow, Lynnwood, WA (US)

Correspondence Address: PATRICK M. DWYER, PROTEOTECH, INC., SUITE 114, 1818 WESTLAKE AVENUE N, SEATLE, WA, 98109, US

(21) Appl. No.: 09/753,313

(22) Filed: Dec. 29, 2000

Related U.S. Application Data
(60) Provisional application No. 60/173,959, filed on Dec. 30, 1999.

(51) Int. Cl. A61K 35/78; A61K 31/7048

(52) U.S. Cl. 424/729; 514/27

(57) ABSTRACT

Green tea and other natural and synthetic sources of catechins, and bioflavanoids, flavanols, flavandiols, flavanoids, and tannins or derivatives thereof, are disclosed for the preparation of a pharmaceutical composition or dietary supplement for the treatment, prevention or management of amyloidosis in a mammalian subject susceptible to, or afflicted by, such a disease. Use of the green tea and its constituents and methods of use are also disclosed. Methods for promoting, maintaining or enhancing in a patient one or more of the mental or cognitive qualities selected from the group of mental or cognitive qualities consisting of mental acuity, mental alertness, cognitive well being, normal brain function, cognitive ability, mental performance, memory, concentration, mental sharpness, mental clarity, short term memory, normal brain function, and learning, the method comprising the step of administering to the patient a therapeutically effective amount of plant matter from a plant of the genus Camellia, species sinensis are also disclosed.

### TECHNICAL FIELD

The invention relates to compositions and methods for treating Alzheimer's Disease and other amyloidoses, and to methods for isolating pharmaceutical agents from plant matter, more particularly, it relates to uses, compositions and methods for therapeutic intervention in Alzheimer's disease and other amyloidoses and in Lewy body and Parkinson's disease using plant matter and derivatives thereof.

### BACKGROUND OF THE INVENTION

Alzheimer's disease is characterized by the accumulation of a 39-43 amino acid peptide termed the beta-amyloid protein or Aβ, in a fibrillar form, existing as extracellular amyloid plaques and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of Alzheimer's disease. Accumulating evidence implicates amyloid as a major causative factor of Alzheimer's disease pathogenesis.

A variety of other human diseases also demonstrate amyloid deposition and usually involve systemic organs (i.e. organs or tissues lying outside the central nervous system), with the amyloid accumulation leading to organ dysfunction or failure. In Alzheimer's disease and “systemic” amyloid diseases, there is currently no cure or effective treatment, and the patient usually dies within 3 to 10 years from disease onset.

Parkinson's disease is also a neurodegenerative disorder, and it is pathologically characterized by the presence of intracytoplasmic Lewy bodies, the major components of which are filaments consisting of alpha-synuclein. Two dominant mutations in alpha-synuclein causing familial early onset Parkinson's disease have been described suggesting that Lewy bodies contribute mechanistically to the degeneration of neurons in Parkinson's disease. Alpha-synuclein fibril formation resembles that of Alzheimer's beta-amyloid protein (Aβ) fibrils. Parkinson's disease alpha-synucleinfibrils, like the Aβ fibrils of Alzheimer's disease, also consist of a predominant beta-pleated sheet structure.

Discovery and identification of new compounds or agents as potential therapeutic agents to arrest amyloid deposition, accumulation and/or persistence that occurs in Alzheimer's disease and other amyloidoses, and in Lewy body and Parkinson's disease, are desperately sought.

### DISCLOSURE OF THE INVENTION

The invention relates to the identification and use of standardized green tea extract and derivatives and constituents thereof for the therapeutic intervention of Alzheimer's disease and other amyloidoses and Parkinson's and Lewy body diseases. In addition, methods of isolation for the identification and purification of the potent amyloid inhibitory ingredients within green tea extract are disclosed. Use of standardized green tea leaf extract and its ingredients (i.e. 50% polyphenols) contained within different commercial preparations are anticipated to benefit human patients with Alzheimer's disease and other amyloidoses and Parkinson's and Lewy body diseases, due to green tea leaf extract's ability to inhibit amyloid fibril formation and Parkinson's α-synuclein fibril and Lewy body formation, and to cause dissolution/disruption, and disaggregation of pre-formed amyloid and α-synuclein fibrils.

### SUMMARY OF THE INVENTION

The present invention pertains to the identification and surprising discovery that the standardized green tea leaf extract (i.e. standardized to 50% polyphenols) acts as an impressive inhibitor of Alzheimer's disease amyloid formation. Furthermore, standardized green tea leaf extract has the ability to cause a disassembly/disruption of pre-formed amyloid fibrils of the Alzheimer's type, suggesting that this agent may be useful for patients at latter stages Alzheimer's disease, and those affected with other amyloid diseases. Standardized green tea leaf extract obtained from different commercial sources (extracts isolated from gelatin-coated capsules) were found to serve as potent inhibitors of Alzheimer's disease amyloid fibrillogenesis.

For purposes of this disclosure, Parkinson's disease, due to the fact that fibrils develop in the brains of patients with this disease (which are Congo red and Thioflavin S positive, and which contain predominant beta-pleated sheet secondary structure), should be regarded as a disease which also displays the characteristics of an amyloid-like disease, and disclosures and claims herein related to amyloidoses are expected in like manner to relate therapeutically to Parkinson's and Lewy body diseases. Therefore agents or compounds found to inhibit Alzheimer's disease Aβ amyloid fibril formation, are anticipated to also be effective in the inhibition of alpha-synuclein fibril formation. These agents or compounds will therefore also serve as therapeutics for Parkinson's and Lewy body disease, in addition to having efficacy as a therapeutic for Alzheimer's disease and other amyloid disorders.

Commercially available standardized green tea leaf extract caused a marked significant dose-dependent inhibition of Aβ 1-40 amyloid fibril formation as determined using a Thioflavin T fluorometry assay in a dose-dependent manner. Standardized green tea leaf extract obtained from commercial sources was also a potent disrupter of pre-formed Aβ 1-42 containing amyloid fibrils, as determined using a Thioflavin T fluorometry assay, and exerted its effects in a dose-dependent manner. Lastly, standardized green tea leaf extract obtained from different commercial sources caused a disaggregation of pre-formed Aβ 1-42 Alzheimer's amyloid fibrils. Therefore, the present invention claims the use of standardized green tea leaf extract (in various forms, i.e. a pill, tablet, liquid form, powder form, etc.) and derivatives thereof from different commercial sources for the treatment of amyloidosis in Alzheimer's disease, type II diabetes and other amyloidoses. Also disclosed are methods of isolation to identify and purify the key amyloid inhibitory ingredients within the green tea extract material. Identification of the “active” amyloid inhibitory ingredients within the green tea extracted plant materials are anticipated to lead to new drug design for anti-amyloid therapeutics of the future. Current use of standardized green tea leaf extract and its ingredients contained within different commercial preparations are anticipated to benefit human patients at all stages of Alzheimer's disease due to standardized green tea extract's newly demonstrated ability to inhibit Aβ amyloid fibril formation (early to mid-stage Alzheimer's disease), and cause dissolution/disruption and disaggregation of pre-formed amyloid fibrils (mid to late stages of Alzheimer's disease). Similarly, standardized green tea leaf extract is anticipated to benefit patients with different systemic amyloid diseases such as type II diabetes, regardless of the stage of amyloid accumulation and the organ (or tissue) involved.

While results are exemplified with Species Camellia sinensis, other species within the family Theaceae are believed to have similar effect.

Features of the Invention

A primary object of the present invention is to establish new methods for the treatment of the amyloid diseases. The amyloid diseases include, but are not limited to, the amyloid associated with Alzheimer's disease, Down's syndrome and hereditary cerebral hemorrhage with amyloidosis of the Dutch type (wherein the specific amyloid is referred to as beta-amyloid protein or Aβ), the amyloid associated with chronic inflammation, various forms of malignancy and Familial Mediterranean Fever (wherein the specific amyloid is referred to as AA amyloid or inflammation-associated amyloidosis), the amyloid associated with multiple myeloma and other B-cell dyscrasias (wherein the specific amyloid is referred to as AL amyloid), the amyloid associated with type II diabetes (wherein the specific amyloid is referred to as amylin or islet amyloid), the amyloid associated with the prion diseases including Creutzfeldt-Jakob disease, Gerstmann-Straussler syndrome, kuru and animal scrapie (wherein the specific amyloid is referred to as PrP amyloid), the amyloid associated with long-term hemodialysis and carpal tunnel syndrome (wherein the specific amyloid is referred to as beta₂-microglobulin amyloid), the amyloid associated with senile cardiac amyloid and Familial Amyloidotic Polyneuropathy (wherein the specific amyloid is referred to as transthyretin or prealbumin), and the amyloid associated with endocrine tumors such as medullary carcinoma of the thyroid (wherein the specific amyloid is referred to as variants of procalcitonin).

Another object of the present invention is to use green tea, green tea leaves and extracts or derivatives thereof, for the treatment of amyloid formation, deposition, accumulation and/or persistence in Alzheimer's disease, type II diabetes and other amyloidoses.

Another object of the present invention is to green tea, green tea leaves and extracts or derivatives thereof, from the Camellia sinensis species for the treatment of amyloid formation, deposition, accumulation and/or persistence in Alzheimer's disease, type II diabetes and other amyloidoses.

Another object of the present invention is to green tea, green tea leaves and extracts or derivatives thereof, from the Theaceae family for the treatment of amyloid formation, deposition, accumulation and/or persistence in Alzheimer's disease, type II diabetes and other amyloidoses.

Another object of the present invention is to use commercially available pills, tablets, caplets, soft and hard gelatin capsules, lozenges, sachets, cachets, vegicaps, liquid drops, elixers, suspensions, emulsions, solutions, syrups, tea bags, tea leaves, aerosols (as a solid or in a liquid medium), suppositories, sterile injectable solutions, sterile packaged powders, and/or leaf powder which contain green tea, green tea leaves and extracts or derivatives thereof, to treat patients with Alzheimer's disease, type II diabetes and other amyloidoses.

Another object of the present invention is to use green tea, green tea leaves and extracts or derivatives thereof, and/or the polyphenols contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the catechins contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the catechins, including but not limited to, catechin, epicatechin, gallocatechin gallate, epigallocatechin gallate, epigallocatechin, and/or epicatechin gallate, whether contained within green tea, green tea leaves and extracts or derivatives thereof, or from other natural or synthetic sources, for the treatment of amyloid formation, deposition, accumulation and/or persistence in Alzheimer's disease, type II diabetes and other amyloidoses.

Yet another object of the present invention is to use the bioflavanoids contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the flavanols contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the flavandiols contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the flavanoids contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to use the tannins contained within green tea, green tea leaves and extracts or derivatives thereof, for the treatment of amyloid formation, deposition, accumulation and/or persistence in Alzheimer's disease, type II diabetes and other amyloidoses.

Yet another object of the present invention is to provide methods to isolate the active ingredients present within green tea, green tea leaves and extracts or derivatives thereof, for use as potent agents which inhibit amyloid formation, amyloid deposition, amyloid accumulation, amyloid persistence, cause a disassembly/disruption and/or cause a disassembly of pre-formed or pre-deposited amyloid fibrils in Alzheimer's disease, type II diabetes and other amyloidoses. Methods for isolation of the active ingredients within green tea, green tea leaves and extracts or derivatives thereof, include application of some standard techniques known to those skilled in the art, including, but not limited to, thin layer chromatography using silica-coated plates, and separation and isolation using high or low pressure liquid chromatography (HPLC). Unknown active ingredients within green tea, green tea leaves and extracts or derivatives thereof, found to be potent inhibitors of amyloid formation, amyloid deposition, amyloid accumulation, amyloid persistence, and/or cause a disassembly/disruption, and disaggregation of pre-formed or pre-depo sited amyloid fibrils in Alzheimer's disease, type II diabetes and other amyloidoses, are identified by re-testing of individual bands or fractions (separated by thin layer chromatography, column chromatography and/or HPLC) using specific assay tests as described in the examples of the present invention. Sufficient isolation of these active ingredients contained within individual bands and/or fractions are then sent out for specific analyses which may include, but are not limited to, scanning electron microscope equipped with energy dispersive x-ray analyzer to detect and spatially map some elements present in each sample, elemental analysis by combustion to determine the relative % of carbon, hydrogen and nitrogen, high resolution mass spectroscopy to determine molecular weight and elemental composition, Fourier transform infrared spectroscopy to determine functional groups and make comparisons to the spectra of known compounds, differential scanning calorimetry to determine melting point, atomic absorption, gel chromatography, high performance liquid chromatography, proton and C¹³ nuclear magnetic resonance spectroscopy for material characterization and to provide information regarding the position of atoms relative to each other, and UV/VIS spectroscopy. It is expected that additional techniques will be developed as part of the further isolation of potent active ingredients within green tea, green tea leaves and extracts or derivatives thereof

Yet another object of the present invention is to provide the use of green tea, green tea leaves and extracts or derivatives thereof, and/or its ingredients [(regardless of commercial source and regardless of final form for consumption by humans, i.e. pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, and/or tea leafpowder] for inhibition of amyloid formation, deposition, accumulation, and/or persistence, regardless of its clinical setting.

Yet another object of the present invention is to provide compositions and methods involving administering to a subject a therapeutic dose of green tea, green tea leaves and extracts or derivatives thereof, which inhibits amyloid deposition. Accordingly, the compositions and methods of the invention are useful for inhibiting amyloidosis in disorders in which amyloid deposition occurs. The compounds of the invention can be used therapeutically to treat amyloidosis or can be used prophylactically in a subject susceptible to amyloidosis. The methods of the invention are based, at least in part, in directly inhibiting amyloid fibril formation, causing disassembly/disruption and/or disaggregation of pre-formed amyloid fibrils.

Yet another object of the present invention is to provide pharmaceutical compositions for treating amyloidosis. The pharmaceutical compositions include a therapeutic compound of the invention in an amount effective to inhibit amyloid deposition and a pharmaceutically acceptable vehicle.

Yet another object of the present invention is the use of any and all synthetic compounds that are similar to green tea, green tea leaves, extracts or derivatives thereof and/or its active ingredients, for use as potent agents which inhibit amyloid formation, amyloid deposition, amyloid accumulation, amyloid persistence, cause a disassembly/ disruption, and/or disaggregation of pre-formed or pre-deposited amyloid fibrils in Alzheimer's disease, type II diabetes and other amyloidoses.

In a particular aspect of the invention there is a method of isolation to purify and identify the amyloid inhibitory ingredients from green tea, green tea leaves and extracts or derivatives thereof. In one such method, an extract prepared from commercially obtained pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, tea powder, using the method employing some or all of the following steps: a) extraction from green tea, green tea leaves and extracts or derivatives thereof regardless of form as described above using water or alcohol (i.e. methanol, ethanol or propanol, b) centrifugation at 2,500×g for 20 minutes and collection of supernatant, c) rotary evaporation to dryness for alcohol-extracted compounds and lyophilization for water-extracted compounds, d) washing dry powder obtained with 4 volumes of petroleum ether (repeated 4 times), followed by centrifugation (each time) at 2,500×g for 20 minutes, and collection of supernatants and pellets, e) air-drying of collected pellets, f) re-extraction of air-dried pellets with water and centrifugation at 2,500×g for 20 minutes, g) lyophilization of collected supernatants (referred to as water extracts), h) re-dissolving the pellets or lyophilized water-extract powder in acetonitrile/water/trifluoroacetic acid (TFA), i) injecting and separation by HPLC or low pressure chromatography, j) identifying amyloid inhibitory ingredients by testing in relevant in vitro and in vivo assays, and k) sending out for structural analysis and elemental composition, as described herein.

Another object of the present invention is to provide a composition, preferably in the form of a dietary supplement, for providing, supporting or improving in a subject one or more of the mental or cognitive qualities selected from the group of mental or cognitive qualities consisting of nutritional support for age related cognitive or memory decline, normal brain function, cognitive ability, and concentration, wherein the composition comprises green tea, green tea leaves and extracts or derivatives thereof.

A further object of the invention is to provide a composition, preferably in the form of a dietary supplement, for promoting, maintaining or enhancing in a subject one or more of the mental or cognitive qualities selected from the group of mental or cognitive qualities consisting of mental acuity, mental alertness, cognitive well being, normal brain function, cognitive ability, mental performance, memory concentration, mental sharpness, mental vitality, mental clarity, short term memory, normal brain function, learning, and good brain health, wherein the composition comprises green tea, green tea leaves and extracts or derivatives thereof.

Still another object of the invention is to provide a composition, preferably in the form of a dietary supplement, for promoting or supporting healthy pancreatic function in a subject, by helping to promote normal insulin function, or for reducing, disrupting, dissolving, inhibiting, eliminating or preventing in a subject one or more conditions involving the pancreas selected from the group of conditions involving the pancreas consisting of amyloid fibril deposits, amyloid protein deposits, pancreas associated with amyloid fibril deposits, pancreas associated amyloid protein deposits, amyloid fibril formation and growth, and pancreas associated amyloid fibril formation and growth, wherein the composition comprises green tea, green tea leaves and extracts or derivatives thereof.

It is yet another object of the invention to meet any or all of the needs summarized above.

In other aspects of the invention, a pharmaceutical agent is disclosed for treating an amyloid disease in a patient, wherein the pharmacological agent comprises a therapeutically effective amount of plant matter from a plant of the family Theraceae, and in particular the genus Camellia. The pharmacological agent is preferably from a plant of the genus Camellia, species sinensis. The pharmacological agent is preferably an extract obtained from Camellia sinensis, the extract being from the dried leaves, and advantageously taken from some commercially available source, such as pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, and/or tea leaf powder.

The pharmacological agent preferably has a therapeutically effective amount of standardized green tea leaf extract in a dosage in the range of from about 0.1 to about 500 mg/kg of body weight of the patient, and more preferably in the range from about 1.0 to about 100 mg/kg of body weight of the patient.

The amyloid disease for treatment with the pharmacological agent is selected from the group consisting of the amyloid associated with Alzheimer's disease, Down's syndrome and hereditary cerebral hemorrhage with amyloidosis of the Dutch type (wherein the specific amyloid is referred to as beta-amyloid protein or Aβ), the amyloid associated with chronic inflammation, various forms of malignancy and Familial Mediterranean Fever (wherein the specific amyloid is referred to as AA amyloid or inflammation-associated amyloidosis), the amyloid associated with multiple myeloma and other B-cell dyscrasias (wherein the specific amyloid is referred to as AL amyloid), the amyloid associated with type II diabetes (wherein the specific amyloid is referred to as amylin or islet amyloid), the amyloid associated with the prion diseases including Creutzfeldt-Jakob disease, Gerstmann-Straussler syndrome, kuru and animal scrapie (wherein the specific amyloid is referred to as PrP amyloid), the amyloid associated with long-term hemodialysis and carpal tunnel syndrome (wherein the specific amyloid is referred to as beta₂-microglobulin amyloid), the amyloid associated with senile cardiac amyloid and Familial Amyloidotic Polyneuropathy (wherein the specific amyloid is referred to as transthyretin or prealbumin), and the amyloid associated with endocrine tumors such as medullary carcinoma of the thyroid (wherein the specific amyloid is referred to as variants of procalcitonin).

Preferred pharmaceutical agents have a weight percentage of plant extract in the agent is in the range of from about 70% to about 95%, and may also have a pharmaceutically acceptable carrier, diluent or excipient. The pharmaceutical agent preferably has an amyloid inhibitory activity or efficacy greater than 50%.

In addition, a composition comprised of green tea, green tea leaves, extracts or derivatives thereof, and plant matter from at least one plant selected from the group of plants consisting of, and commonly known as Cat's claw, ginkgo biloba, rosemary, gotu kola, bacopin, and ginseng has the ability to inhibit the formation of brain amyloid deposits in subjects who accumulate brain amyloid deposits that occur during normal aging and in a variety of brain disorders including Alzheimer's disease; it will therefore promote mental alertness in such subjects.

Compositions of the invention also have the ability to reduce, eliminate, prevent, inhibit, disrupt, disassemble, or disaggregate amyloid fibril or protein deposits, brain associated amyloid fibril deposits or brain associated amyloid protein deposits, as well as amyloid fibril formation, or age associated amyloid fibril formation, brain associated amyloid fibril formation; it will therefore promote mental acuity, promote mental alertness, provide nutritional support for age or related cognitive or memory decline, promote cognitive well being, support brain function, improve cognitive ability, mental performance or memory, promote concentration and mental sharpness, improve mental vitality, promote greater mental clarity and alertness, improve short term memory, reduce or reverse age associated cognitive or memory decline, support normal brain function , enhance learning or memory; improve concentration, enhance mental performance, reduce mental decline, reduce likelihood of age related brain disorders, and maintain good brain health.

It is anticipated that compositions of the invention also have the ability to reduce, eliminate, prevent, inhibit, disrupt, disassemble or disaggregate amyloid fibril or protein deposits, pancreas associated amyloid fibril or protein deposits, as well as amyloid fibril formation and growth, and pancreas associated amyloid fibril formation and growth; it will therefore support healthy pancreatic function and promote pancreatic function by helping to promote normal insulin function.

Compositions of the invention may also include carriers, diluents and/or excipients commonly used in the pharmaceutical and dietary supplement industries and any such additions as will be known to those skilled in the art are acceptable and may be employed without departing from the scope of the invention.

Another aspect of the invention is a method for isolating amyloid inhibitory constituents within green tea, green tea leaves, extracts or derivatives thereof, the method comprising the following steps: a) extracting the green tea, green tea leaves, extracts or derivatives thereof with water, or with an organic solvent, b) removing insoluble materials, c) evaporation to dryness or lyophilization to obtain powder, d) recovering and redissolving the amyloid inhibitory constituents obtained in the water or organic solvent, and e) injecting and separation by high pressure or low pressure liquid chromatography.

Representative constituents of green tea include catechins, bioflavanoids, flavanols, flavandiols, flavanoids, tannins or derivatives thereof, although for purposes of this disclosure these substance may optionally also be synthetically derived or independently found in other plant sources.

The plant matter is preferably comprised of commercially obtained pills, tablets, caplets, soft and hard gelatin capsules, lozenges, sachets, cachets, vegicaps, liquid drops, elixers, suspensions, emulsions, solutions, syrups, tea bags, aerosols (as a solid or in a liquid medium), suppositories, sterile injectable solutions, sterile packaged powders, and/or tea powder, which contain green tea, green tea leaves and extracts or derivatives thereof, and may be taken from commercially available gelatin-coated capsules which contain dried-powder of green tea, green tea leaves, and extracts or derivatives thereof.

The step of extracting the plant matter with an organic solvent further comprises adding methanol initially to plant materials that are powdered, and the resulting mixture is stirred overnight. The solvent used in the step of extracting amyloid inhibitory ingredients preferably has a polarity ranging from that of water to that of pentanol. The step of removing insoluble materials is preferably effected by centrifuging the extract and collecting the supernatant. The step of concentrating the extract is preferably effected by rotary evaporation or lyophilization (for water extracts). Following the extraction and centrifugation steps, the extraction and centrifugation procedure is preferably repeated 1 to 5 more times and the supernatants are collected.

Following the repeated steps of extraction and concentration, the supernatants are preferably pooled and dried using a rotary evaporator or lyophilization (for water extracts). The dry powder is washed with 4 volumes of petroleum ether (repeated 4 times), followed by centrifugation (each time) at 2,500×g for 20 minutes, and collection of the supernatants and pellets. The collected pellets are air-dried and re-extracted with water and centrifuged at 2,500×g for 20 minutes. The collected supernatants (referred to as water extracts) are lyophilized, and the pellets or lyophilized water-extract powder are re-dissolved in acetonitrile/water/trifluoroacetic acid (TFA) for HPLC injection or low pressure chromatography. The dissolved pellet is divided into equal portions and injected into an HPLC. The HPLC preferably contains a 1×25 cm C₁₈ column, though other sizes may be made to serve, and is maintained at 30° C. with a flow rate of 2 ml/min. The sample portions injected onto the HPLC are eluted with gradients of A and B, such that 0% B for 5 minutes, 0-15% B from 5-10 minutes, 15-45% B from 10-70 minutes, and 45-100% B from 70-85 minutes; where B=95% acetonitrile with 0.5% acetic acid in distilled water and A=5% acetonitrile with 0.5% acetic acid in distilled water. The eluents from the HPLC are monitored at all wavelengths and 4 ml fractions are collected in a fraction collector and pooled peaks are obtained at various retention times (from 0 through 85 minutes). The fractions obtained may be concentrated by lyophilization after most of the acetonitrile is removed by rotary evaporation.

The concentrated fractions obtained are then tested in relevant in vitro assays to identify potent inhibitors of amyloid fibril formation, or disassembly/disruption or disaggregation of pre-formed amyloid fibrils. The amyloid inhibitory ingredients are preferably drawn from the HPLC approximate retention times of 10-70 minutes.

A method is also disclosed for treating an amyloid disease in a patient comprising the step of administering to the patient a therapeutically effective amount of green tea, green tea leaves and extracts or derivatives thereof The green tea, green tea leaves, and extracts or derivatives thereof, are preferably administered orally or by aerosol spray or in a parentally injectable or infusible form.

In the methods of the invention, amyloid formation, deposition, accumulation and/or persistence in a subject is inhibited by administering a therapeutic dose of the invention to the subject. The term subject is intended to include living organisms in which amyloidosis can occur. Examples of subjects include humans, monkeys, cows, sheep, goats, dogs, cats, mice, rats and transgenic species thereof. Administration of green tea, green tea leaves and extracts or derivatives thereof, to a subject to be treated can be carried out using known procedures, at dosages and for periods of time effective to inhibit amyloid formation, deposition, accumulation and persistence in the subject. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to inhibit amyloidosis in the subject.

Representative Method or Process Embodiments

1. A method of treatment, prevention, or management of an amyloidosis in a mammalian subject susceptible to, or afflicted by, the amyloidosis is presented, the method comprising the step of administering to the subject a therapeutic amount of plant matter from a source of green tea, green tea leaves, standardized green tea extract, or green tea derivative.

The amyloidosis in any of these embodiments is preferably selected from the group of amyloidoses consisting of Alzheimer's disease, type II diabetes, Down's syndrome, hereditary cerebral hemorrhage with amyloidosis of the Dutch type, the amyloidosis associated with chronic inflammation, various forms of malignancy and familial Mediterranean fever, the amyloidosis associated with multiple myeloma and other B-cell dyscrasias, the amyloidosis associated with type II diabetes, the amyloidosis associated with the prion diseases including Creutzfeldt-Jakob disease, Gerstmann-Straussler syndrome, kuru and animal scrapie, the amyloidosis associated with long-term hemodialysis and carpal tunnel syndrome, the amyloidosis associated with senile cardiac amyloid and familial amyloidosis polyneuropathy, and the amyloidosis associated with endocrine tumors such as medullary carcinoma of the thyroid.

2. A method for the treatment, inhibition, prevention or management of amyloid formation, deposition, accumulation, aggregation and/or persistence in Alzheimer's disease, type II diabetes and other amyloidoses in a mammalian subject is presented, the method comprising the step of administering to the subject a therapeutic amount of a substance selected from the group of substances consisting of green tea, green tea leaves, standardized green tea extract, green tea derivative, catechins, bioflavanoids, flavanols, flavandiols, flavanoids, tannins or derivatives thereof

The substance is preferably a catechin selected from the group of catechins consisting of catechin, epicatechin, gallocatechin gallate, epigallocatechin gallate, epigallocatechin, and epicatechin gallate, or a derivative of one of the above group.

3. A method for the treatment, prevention or management of an amyloidosis in a mammalian subject susceptible to the amyloidosis is presented, the method comprising the step of administering to the subject a therapeutic amount of a substance selected from the group of substances consisting of catechins, bioflavanoids, flavanols, flavandiols, flavanoids, tannins or derivatives thereof.

The substance is preferably a catechin selected from the group of catechins consisting of catechin, epicatechin, gallocatechin gallate, epigallocatechin gallate, epigallocatechin, and epicatechin gallate, or a derivative of one of the above group.

In any of these embodiments, within the step of administering plant matter, a therapeutic quantity of one or more plant materials selected from the group of plants consisting of, and commonly known as, Cat's claw, ginkgo biloba, rosemary, gotu kola, bacopin, and ginseng may also optionally be administered.

4. A method for the treatment, inhibition, prevention or management of α-synuclein fibril formation, deposition, accumulation, aggregation and/or persistence in Parkinson's disease or Lewy body disease in a mammalian subject is presented, the method comprising the step of administering to the subject a therapeutic amount of a substance selected from the group of substances consisting of green tea, green tea leaves, standardized green tea extract, green tea derivative, catechins, bioflavanoids, flavanols, flavandiols, flavanoids, tannins or derivatives thereof

The substance is preferably a catechin selected from the group of catechins consisting of catechin, epicatechin, gallocatechin gallate, epigallocatechin gallate, epigallocatechin, and epicatechin gallate, or a derivative of one of the above group.

5. A method for promoting mental alertness in a patient is presented, the method comprising the step of administering to the patient a therapeutically effective amount of plant matter from a plant of the family Theaceae, and preferably from a plant of the genus Camellia, species sinensis. This method may also be used for inhibiting the formation of brain amyloid deposits.

6. A method for promoting, maintaining or enhancing in a patient one or more of the mental or cognitive qualities selected from the group of mental or cognitive qualities consisting of mental acuity, mental alertness, cognitive well being, normal brain function, cognitive ability, mental performance, memory, concentration, mental sharpness, mental clarity, short term memory, normal brain function, and learning is presented, the method comprising the step of administering to the patient a therapeutically effective amount of plant matter from a plant of the genus Camellia, species sinensis.

7. A method for providing, supporting or improving in a patient one or more of the mental or cognitive qualities selected from the group of mental or cognitive qualities consisting of normal brain function, cognitive ability, and concentration is presented, the method comprising the step of administering to the patient a therapeutically effective amount of plant matter from a plant of the genus Camellia, species sinensis.

8. A method for reducing in a patient one or more of the mental or cognitive effects selected from the group of mental or cognitive effects consisting of, age associated cognitive or memory decline, mental decline, and likelihood of age related brain or cognitive disorders is presented, the method comprising the step of administering to the patient a therapeutically effective amount of plant matter from a plant of the genus Camellia, species sinensis.

9. A method for reducing, disrupting, dissolving, inhibiting, eliminating or preventing in a patient one or more conditions involving the brain selected from the group of conditions involving the brain consisting of amyloid fibril deposits, amyloid protein deposits, brain associated amyloid fibril deposits, brain associated amyloid protein deposits, amyloid fibril formation and growth, age associated amyloid fibril formation and growth, brain associated amyloid fibril formation and growth is presented, the method comprising the step of administering to the patient a therapeutically effective amount of plant matter from a plant of the genus Camellia, species sinensis.

10. A method for promoting or supporting healthy pancreatic function in a patient, by helping to promote normal insulin function is presented, the method comprising the step of administering to the patient a therapeutically effective amount of plant matter from a plant of the genus Camellia, species sinensis.

11. A method for reducing, disrupting, dissolving, inhibiting, eliminating or preventing in a patient one or more conditions involving the pancreas selected from the group of conditions involving the pancreas consisting of amyloid fibril deposits, amyloid protein deposits, pancreas associated amyloid fibril deposits, pancreas associated amyloid protein deposits, amyloid fibril formation and growth, pancreas associated amyloid fibril formation and growth is presented, the method comprising the step of administering to the patient a therapeutically effective amount of plant matter from a plant of the genus Camellia, species sinensis.

Representative Use and/or Composition/agent Embodiments

1. The use of a source of green tea, green tea leaves or standardized green tea leaf extract or derivatives thereof in the preparation of a pharmaceutical composition or dietary supplement for the treatment, prevention and or management of an amyloidosis in a mammalian subject susceptible to, or afflicted by, the amyloidosis is presented.

2. The use of a source of green tea, green tea leaves, standardized green tea leaf extract or derivatives thereof in the preparation of a pharmaceutical composition or dietary supplement for inhibiting amyloid fibril formation, deposition, accumulation, or persistence or causing dissolution/disruption or disaggregation of pre-formed amyloid fibrils is presented.

3. A pharmaceutical composition or dietary supplement for the treatment, prevention, or management of amyloidosis in a mammalian subject susceptible to, or afflicted by, the amyloidosis is presented, the composition comprising a source of green tea, green tea leaves or standardized green tea leaf extract or derivatives thereof, and, if desired, a pharmaceutically or dietarily acceptable carrier, diluent or excipient.

4. A pharmaceutical composition or dietary supplement for inhibiting amyloid fibril formation, deposition, accumulation, or persistence or causing dissolution/disruption and or disaggregation of pre-formed amyloid fibrils is presented, the composition comprising a source of green tea, green tea leaves or standardized green tea leaf extract or derivatives thereof and, if desired, a pharmaceutically or dietarily acceptable carrier, diluent or excipient.

5. The use of catechins, bioflavanoids, flavanols, flavandiols, flavanoids, tannins or derivatives thereof for the treatment, prevention or management of amyloid formation, deposition, accumulation and/or persistence in Alzheimer's disease, type II diabetes and other amyloidoses in a mammalian subject susceptible to the amyloidosis is presented.

6. A pharmaceutical composition or dietary supplement for the treatment, prevention or management of amyloid formation, deposition, accumulation and/or persistence in Alzheimer's disease, type II diabetes and other amyloidoses in a mammalian subject susceptible to such amyloid condition is presented, the composition comprising catechins, bioflavanoids, flavanols, flavandiols, flavanoids, tannins or derivatives thereof and, if desired, a pharmaceutically or dietarily acceptable carrier, diluent or excipient.

7. A pharmaceutical composition or dietary supplement for the treatment, inhibition, prevention or management of α-synuclein fibril formation, deposition, accumulation, aggregation and/or persistence in Parkinson's disease or Lewy body disease in a mammalian subject is presented, the composition comprising a substance selected from the group of substances consisting of green tea, green tea leaves, standardized green tea extract, green tea derivative, catechins, bioflavanoids, flavanols, flavandiols, flavanoids, tannins or derivatives thereof.

For use or composition 1, 3 or 6 the amyloidosis is preferably Alzheimer's disease, type II diabetes, or another amyloidosis such as Down's syndrome and hereditary cerebral hemorrhage with amyloidosis of the Dutch type, the amyloidosis associated with chronic inflammation, various forms of malignancy and familial Mediterranean fever, the amyloidosis associated with multiple myeloma and other B-cell dyscrasias, the amyloidosis associated with the prion diseases including Creutzfeldt-Jakob disease, Gerstmann-Straussler syndrome, kuru and animal scrapie, the amyloidosis associated with long-term hemodialysis and carpal tunnel syndrome, the amyloidosis associated with senile cardiac amyloid and familial amyloidosis polyneuropathy, and the amyloidosis associated with endocrine tumors such as medullary carcinoma of the thyroid.

For use or composition 2, 4 or 5 the affected amyloid is preferably beta-amyloid protein or Aβ, AA amyloid or inflammation-associated amyloid, AL amyloid, amylin or islet amyloid, PrP amyloid, beta₂-microglobulin amyloid, transthyretin or prealbumin, or variants of procalcitonin.

For use or composition 1, 2, 3, 4 or 7 the green tea source preferably comprises a commercially available source such as pills, tablets, caplets, soft and hard gelatin capsules, lozenges, sachets, cachets, vegicaps, liquid drops, elixirs, suspensions, emulsions, solutions, syrups, tea bags, tea leaves, aerosols (as a solid or in a liquid medium), suppositories, sterile injectable solutions, sterile packaged powders, and/or leaf powder which source contains green tea, green tea leaves or extracts or derivatives thereof

For use or composition 1, 2, 3, 4 or 7 the extract is preferably obtained from a plant of the Species Camellia sinensis or from another plant of the Theaceae family.

For use or composition 5 or 6 the catechins, bioflavanoids, flavanols, flavandiols, flavanoids , tannins or derivatives thereof are preferably contained within a source of green tea, green tea leaves and extracts or derivatives thereof.

For use or composition 5, 6 or 7 the catechin is preferably a member selected from the group consisting of catechin, epicatechin, gallocatechin gallate, epigallocatechin gallate, epigallocatechin, and epicatechin gallate, or a derivative of one of the above group.

8. The use of a source of green tea, green tealeaves or standardized green tea leaf extract or derivatives thereof in the preparation of a pharmaceutical composition or dietary supplement for providing, supporting or improving in a subject one or more of the mental or cognitive qualities is presented.

9. The use of a source of green tea, green tea leaves or standardized green tea leaf extract or derivatives thereof in the preparation of a pharmaceutical composition or dietary supplement for promoting or supporting healthy pancreatic function in a subject is presented.

10. A pharmaceutical composition or dietary supplement for providing, supporting or improving in a subject one or more of the mental or cognitive qualities which comprises a source of green tea, green tea leaves or standardized green tea leaf extract or derivatives thereof is presented.

11. A pharmaceutical composition or dietary supplement for promoting or supporting healthy pancreatic function in a subject which comprises a source of green tea, green tea leaves or standardized green tea leaf extract or derivatives thereof is presented.

12. A pharmacological agent for promoting, maintaining or enhancing in a patient one or more of the mental or cognitive qualities selected from the group of mental or cognitive qualities consisting of mental acuity, mental alertness, cognitive well being, normal brain function, cognitive ability, mental performance, memory, concentration, mental sharpness, mental vitality, mental clarity, short-term memory, normal brain function, and learning, and good brain health is presented, wherein the pharmacological agent comprises a therapeutically effective amount of plant matter from a plant of the genus Camellia, species sinensis.

For pharmaceutical composition or dietary supplement 2, 3, 6, 10, 11 or 12 one or more additional plant materials selected from the group of plants consisting of, and commonly known as Cat's claw, ginkgo biloba, rosemary, gotu kola, bacopin, and ginseng may optionally be combined to inhibit the formation of brain amyloid deposits in subjects who accumulate brain amyloid deposits that occur during normal aging and in a variety of brain disorders including Alzheimer's disease.

For pharmacological agent 12 a therapeutically effective dosage is optimally in the range of from about 10 to 1,000 mg/kg of body weight, but more preferably in the range of about 10 to 100 mg/kg of body weight of the patient.

13. A pharmacological agent for providing, supporting or improving in a patient one or more of the mental or cognitive qualities selected from the group of mental or cognitive qualities consisting of nutritional support for age related cognitive or memory decline, normal brain function, cognitive ability, and concentration is presented, wherein the pharmacological agent comprises a therapeutically effective amount of plant matter from a plant of the genus Camellia, species sinensis.

14. A pharmacological agent for reducing in a patient one or more of the mental or cognitive effects selected from the group of mental or cognitive effects consisting of, age associated cognitive or memory decline, mental decline, and likelihood of age related brain or cognitive disorders is presented, wherein the pharmacological agent comprises a therapeutically effective amount of plant matter from a plant of the genus Camellia, species sinensis.

15. A pharmacological agent for reducing, disrupting, dissolving, inhibiting or preventing in a patient one or more conditions involving the brain selected from the group of conditions involving the brain consisting of amyloid fibril deposits, amyloid protein deposits, brain associated amyloid fibril deposits, Aβ brain deposits, brain associated Aβ deposits, brain associated amyloid protein deposits, brain amyloid deposits, amyloid fibril formation and growth, age associated amyloid fibril formation and growth is presented, wherein the pharmacological agent comprises a therapeutically effective amount of plant matter from a plant of the genus Camellia, species sinensis.

16. A pharmacological agent for promoting or supporting healthy pancreatic function in a patient, by helping to promote normal insulin function is presented, wherein the pharmacological agent comprises a therapeutically effective amount of plant matter from a plant of the genus Camellia, species sinensis.

17. A pharmacological agent for reducing, disrupting, dissolving, inhibiting or eliminating or preventing in a patient one or more conditions involving the pancreas selected from the group of conditions involving the pancreas consisting of amyloid fibril deposits, amyloid protein deposits, pancreas associated amyloid fibril deposits, amylin deposits, islet amyloid polypeptide deposits, pancreas associated amyloid protein deposits, amyloid fibril formation and growth, pancreas associated amyloid fibril formation and growth is presented, wherein the pharmacological agents comprises a therapeutically effective amount of plant matter from a plant of the genus Camellia, species sinensis.

These and other features and advantages of the present invention will become more fully apparent when the following detailed description of the invention is read in conjunction with the accompanying figures.

### BRIEF DESCRIPTION OF THE DRAWINGS

FIG. 1 is a black and white graph of a Thioflavin T fluorometry assay utilized to determine the dose-dependent effects of standardized green tea leaf extract on inhibition of Alzheimer's Aβ 1-40 amyloid fibril formation.

FIG. 2 is a black and white graph of a Thioflavin T fluorometry assay utilized to determine the dose-dependent effects of standardized green tea leaf extract on disassembly/disruption of pre-formed Alzheimer's Aβ 1-42 amyloid fibrils.

FIG. 3 is a black and white graph of a Congo red-Aβ spectrophotometric assay to determine the effects of standardized green tea leaf extract (from 2 commercial sources) on disaggregation of preformed Alzheimer's Aβ 1-42 fibrils.

### BEST MODE OF CARRYING OUT THE INVENTION

Turning now to the Drawings and Examples, the invention will be described in preferred embodiments by detailed reference to them.

Amyloid and Amyloidosis

Amyloid is a generic term referring to a group of diverse, but specific extracellular protein deposits which all have common morphological properties, staining characteristics, and x-ray diffraction spectra. Regardless of the nature of the amyloid protein deposited all amyloids have the following characteristics: 1) an amorphous appearance at the light microscopic level and appear eosinophilic using hematoxylin and eosin stains; 2) all stain with Congo red and demonstrate a red/green birefringence as viewed under polarized light (Puchtler et al., J. Histochem. Cytochem. 10:355-364, 1962), 3) all contain a predominant beta-pleated sheet secondary structure, and 4) ultrastructurally amyloid usually consist of non-branching fibrils of indefinite length and with a diameter of 7-10 nm.

Amyloid today is classified according to the specific amyloid protein deposited. The amyloid diseases include, but are not limited to, the amyloid associated with Alzheimer's disease, Down's syndrome and Hereditary cerebral hemorrhage with amyloidosis of the Dutch type (wherein the specific amyloid is referred to as beta-amyloid protein or Aβ), the amyloid associated with chronic inflammation, various forms of malignancy and Familial Mediterranean Fever (wherein the specific amyloid is referred to as Aβ amyloid or inflammation-associated amyloidosis), the amyloid associated with multiple myeloma and other B-cell dyscrasias (wherein the specific amyloid is referred to as AL amyloid), the amyloid associated with type II diabetes (wherein the specific amyloid is referred to as amylin or islet amyloid), the amyloid associated with the prion diseases including Creutzfeldt-Jakob disease, Gerstmann-Straussler syndrome, kuru and animal scrapie (wherein the specific amyloid is referred to as PrP amyloid), the amyloid associated with long-term hemodialysis and carpal tunnel syndrome (wherein the specific amyloid is referred to as beta₂-microglobulin amyloid), the amyloid associated with senile cardiac amyloid and Familial Amyloidotic Polyneuropathy (wherein the specific amyloid is referred to as prealbumin or transthyretin amyloid), and the amyloid associated with endocrine tumors such as medullary carcinoma of the thyroid (wherein the specific amyloid is referred to as variants of procalcitonin).

Although amyloid deposits in clinical conditions share common physical properties relating to the presence of a beta-pleated sheet conformation, it is now clear that many different chemical types exist and additional ones are likely to be described in the future. It is currently thought that there are several common pathogenetic mechanisms that may be operating in amyloidosis in general. In many cases, a circulating precursor protein may result from overproduction of either intact or aberrant molecules (ex. plasma cell dyscrasias), reduced degradation or excretion (serum amyloid A in some secondary amyloid syndromes and beta₂-microglobulin in long-term hemodialysis), or genetic abnormalities associated with variant proteins (ex. familial amyloidosis polyneuropathy). Proteolysis of a larger protein precursor molecule occurs in many types of amyloidosis, resulting in the production of lower molecular weight fragments that polymerize and assume a beta-pleated sheet conformation as tissue deposits, usually in an extracellular location. What are the precise mechanisms involved, and the aberrant causes leading to changes in proteolytic processing and/or translational modifications is not known in most amyloids.

Systemic amyloids which include the amyloid associated with chronic inflammation, various forms of malignancy and Familial Mediterranean Fever (ie. AA amyloid or inflammation-associated amyloidosis)(Benson and Cohen, Arth. Rheum. 22:36-42, 1979; Kamei et al, Acta Path. Jpn. 32:123-133, 1982, McAdam et al, Lancet 2:572-573, 1975; Metaxas, Kidney Int. 20:676-685, 1981), and the amyloid associated with multiple myeloma and other B-cell dyscrasias (ie. AL amyloid)(Harada et al, J. Histochem. Cytochem. 19:1 15, 1971), as examples, are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in kidney may lead to renal failure, whereas amyloid deposition in heart may lead to heart failure. For these patients, amyloid accumulation in systemic organs leads to eventual death generally within 3-5 years. Other amyloidoses may affect a single organ or tissue such as observed with the Aβ amyloid deposits found in the brains of patients with Alzheimer's disease and Down's syndrome: the PrP amyloid deposits found in the brains of patients with Creutzfeldt-Jakob disease, Gerstmann-Straussler syndrome, and kuru; the islet amyloid (amylin) deposits found in the islets of Langerhans in the pancreas of 90% of patients with type II diabetes (Johnson et al, N. Engl. J. Med. 321:513-518, 1989; Lab. Invest. 66:522 535, 1992); the beta₂-microglobulin amyloid deposits in the medial nerve leading to carpal tunnel syndrome as observed in patients undergoing long-term hemodialysis (Geyjo et al, Biochem. Biophys. Res. Comm. 129:701-706, 1985; Kidney Int. 30:385-390, 1986); the prealbumin/transthyretin amyloid observed in the hearts of patients with senile cardiac amyloid; and the prealbumin/transthyretin amyloid observed in peripheral nerves of patients who have Familial Amyloidotic Polyneuropathy (Skinner and Cohen, Biochem. Biophys. Res. Comm. 99:1326-1332, 1981; Saraiva et al, J. Lab. Clin. Med. 102:590-603, 1983; J. Clin. Invest. 74:104-119, 1984; Tawara et al, J. Lab. Clin. Med. 98:811-822, 1989). Alzheimer's Disease and the Aging Population.

Alzheimer's disease is a leading cause of dementia in the elderly, affecting 5-10% of the population over the age of 65 years (A Guide to Understanding Alzheimer's Disease and Related Disorders, edited by Jorm, New York University Press, New York, 1987). In Alzheimer's disease, the parts of the brain essential for cognitive processes such as memory, attention, language, and reasoning degenerate, robbing victims of much that makes us human, including independence. In some inherited forms of Alzheimer's disease, onset is in middle age, but more commonly, symptoms appear from the mid-60's onward. Alzheimer's disease today affects 4-5 million Americans, with slightly more than half of these people receiving care at home, while the others are in many different health care institutions. The prevalence of Alzheimer's disease and other dementias doubles every 5 years beyond the age of 65, and recent studies indicate that nearly 50% of all people age 85 and older have symptoms of Alzheimer's disease (1997 Progress Report on Alzheimer's Disease, National Institute on Aging/National Institute of Health). 13% (33 million people) of the total population of the United States are age 65 and older, and this % will climb to 20% by the year 2025 (1997 Progress Report on Alzheimer's Disease, National Institute on Aging/National Institute of Health).

Alzheimer's disease also puts a heavy economic burden on society as well. A recent study estimated that the cost of caring for one Alzheimer's disease patient with severe cognitive impairments at home or in a nursing home, is more than $47,000 per year (A Guide to Understanding Alzheimer's Disease and Related Disorders, edited by Jorm, New York University Press, New York, 1987). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (1997 Progress Report on Alzheimer's Disease, National Institute on Aging/National Institute of Health).

Tacrine hydrochloride (“Cognex”) , the first FDA approved drug for Alzheimer's disease is a acetylcholinesterase inhibitor (Cutler and Sramek, N. Engl. J. Med. 328:808 810, 1993). However, this drug has showed limited success in the cognitive improvement in Alzheimer's disease patients and initially had major side effects such as liver toxicity. The second more recently FDA approved drug, donepezil (also known as “Aricept”) , which is also an acetylcholinesterase inhibitor, is more effective than tacrine, by demonstrating slight cognitive improvement in Alzheimer's disease patients (Barner and Gray, Ann. Pharmacotherapy 32:70-77, 1998; Rogers and Friedhoff, Eur. Neuropsych. 8:67-75, 1998), but is not believed to be a cure. Therefore, it is clear that there is a need for more effective treatments for Alzheimer's disease patients.

Amyloid as a Therapeutic Target for Alzheimer's Disease

Alzheimer's disease is characterized by the deposition and accumulation of a 39-43 amino acid peptide termed the beta-amyloid protein, Aβ or β/A4 (Glenner and Wong, Biochem. Biophys. Res. Comm. 120:885-890, 1984; Masters et al, Proc. Natl. Acad. Sci. USA 82:4245-4249, 1985; Husby et al, Bull WHO 71:105-108, 1993). Aβ is derived by protease cleavage from larger precursor proteins termed beta-amyloid precursor proteins (or βPPs) of which there are several alternatively spliced variants. The most abundant forms of the βPPs include proteins consisting of 695,751 and 770 amino acids (Tanzi et al, Nature 331:528-530, 1988; Kitaguchi et al, Nature 331:530-532, 1988; Ponte et al, Nature 331:525-527, 1988).

The small Aβ peptide is a major component which makes up the amyloid deposits of “plaques” in the brains of patients with Alzheimer's disease. In addition, Alzheimer's disease is characterized by the presence of numerous neurofibrillary “tangles”, consisting of paired helical filaments which abnormally accumulate in the neuronal cytoplasm (Grundke-Iqbal et al, Proc. Natl. Acad. Sci. USA 83:4913-4917, 1986; Kosik et al, Proc. Natl. Acad. Sci. USA 83:4044-4048, 1986; Lee et al, Science 251:675-678, 1991). The pathological hallmarks of Alzheimer's disease is therefore the presence of “plaques” and “tangles”, with amyloid being deposited in the central core of plaques. The other major type of lesion found in the Alzheimer's disease brain is the accumulation of amyloid in the walls of blood vessels, both within the brain parenchyma and in the walls of meningeal vessels which lie outside the brain. The amyloid deposits localized to the walls of blood vessels are referred to as cerebrovascular amyloid or congophilic angiopathy (Mandybur, J. Neuropath. Exp. Neurol. 45:79-90, 1986; Pardridge et al, J. Neurochem. 49:1394-1401, 1987).

For many years there has been an ongoing scientific debate as to the importance of “amyloid” in Alzheimer's disease and whether the “plaques” and “tangles” characteristic of this disease, were a cause or merely the consequences of the disease. Within the last few years, studies now indicate that amyloid is indeed a causative factor for Alzheimer's disease and should not be regarded as merely an innocent bystander. The Alzheimer's Aβ protein in cell culture has been shown to cause degeneration of nerve cells within short periods of time (Pike et al, Br. Res. 563:311-314, 1991; J. Neurochem. 64:253-265, 1995). Studies suggest that it is the fibrillar structure (consisting of a predominant β-pleated sheet secondary structure), characteristic of all amyloids, that is responsible for the neurotoxic effects. Aβ has also been found to be neurotoxic in slice cultures of hippocampus (Harrigan et al, Neurobiol. Aging 16:779-789, 1995) and induces nerve cell death in transgenic mice (Games et al, Nature 373:523-527, 1995; Hsiao et al, Science 274:99-102, 1996). Injection of the Alzheimer's Aβ into rat brain also causes memory impairment and neuronal dysfunction (Flood et al, Proc. Natl. Acad. Sci. 88:3363-3366, 1991; Br. Res. 663:271-276, 1994).

Probably, the most convincing evidence that Aβ amyloid is directly involved in the pathogenesis of Alzheimer's disease comes from genetic studies. It has been discovered that the production of Aβ can result from mutations in the gene encoding, its precursor, beta amyloid precursor protein (Van Broeckhoven et al, Science 248:1120-1122, 1990; Murrell et al, Science 254:97-99, 1991; Haass et al, Nature Med. 1:1291-1296, 1995). The identification of mutations in the beta-amyloid precursor protein gene which causes early onset familial Alzheimer's disease is the strongest argument that amyloid is central to the pathogenetic process underlying this disease. Four reported disease-causing mutations have now been discovered which demonstrate the importance of Aβ in causing familial Alzheimer's disease (reviewed in Hardy, Nature Genet. 1:233-234, 1992). All of the se studies suggest that providing a drug to reduce, eliminate or prevent fibrillar Aβ formation, deposition, accumulation and/or persistence in the brains of human patients is believed to serve as an effective therapeutic.

In addition, the alpha-synuclein protein which forms fibrils, and is Congo red and Thioflavin S positive, is found as part of Lewy bodies in the brains of patients with Parkinson's disease (Lewy in Handbuch der Neurologie, M. Lewandowski, ed., Springer, Berline pp.920-933, 1912; Pollanen et al, J. Neurospath. Exp. Neurol. 52:183-191, 1993; Spillantini et al, Proc. Natl. Acad. Sci. USA 95:6469-6473, 1998; Arai et al, Neurosc. Lett. 259:83-86, 1999). For purposes of this disclosure, Parkinson's disease, due to the fact that fibrils develop in the brains of patients with this disease (which are Congo red and Thioflavin S positive, and which contain predominant beta-pleated sheet secondary structure), should be regarded as a disease which also displays the characteristics of an amyloid-like disease.

Parkinson's Disease and Alpha-Synuclein Fibril Formation

Parkinson's disease is a neurodegenerative disorder that is pathologically characterized by the presence of intracytoplasmic Lewy bodies (Lewy in Handbuch der Neuroloegi, M. Lewandowski, ed., Springer, Berline pp.920-933, 1912; Pollanen et al, J. Neuropath. Exp. Neurol. 52:183-191, 1993), the major components of which are filaments consisting of alpha-synuclein (Spillantini et al, Proc. Natl. Acad. Sci. USA 95:6469-6473, 1998; Arai et al, Neurosc. Lett. 259:83-86,1999), an 140-amino acid protein (Ueda et al, Proc. Natl. Acad. Sci. USA 90:11282-11286, 1993). Two dominant mutations in alpha-synuclein causing familial early onset Parkinson's disease have been described suggesting that Lewy bodies contribute mechanistically to the degeneration of neurons in Parkinson's disease (Polymeropoulos et al, Science 276:2045-2047, 1997; Kruger et al, Nat. Genet. 18:106-108, 1998). Recently, in vitro studies have demonstrated that recombinant alpha-synuclein can indeed form Lewy body-like fibrils (Conway et al, Nature Med. 4:1318-1320, 1998; Hashimoto et al, Brain Res. 799:301-306, 1998; Nahri et al, J. Biol. Chem. 274:9843-9846, 1999). Most importantly, both Parkinson's disease-inked alpha-synuclein mutations accelerate this aggregation process which suggests that such in vitro studies may have relevance for Parkinson's disease pathogenesis. Alpha-synuclein aggregation and fibril formation fulfills of the criteria of a nucleation-dependent polymerization process (Wood et al, J. Biol. Chem. 274:19509-19512, 1999). In this regard alpha-synuclein fibril formation resembles that of Alzheimer's beta-amyloid protein (Aβ) fibrils. Alpha-synuclein recombinant protein, and non-amyloid component (known as NAC-P), which is a 35-amino acid peptide fragment of alpha-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al, Brain Res. 799:301-306, 1998; Ueda et al, Proc. Natl. Acad. Sci. USA 90:11282-11286, 1993).

Parkinson's disease alpha-synuclein fibrils, like the Aβ fibrils of Alzheimer's disease, also consist of a predominant beta-pleated sheet structure. Therefore agents or compounds found to inhibit Alzheimer's disease Aβ amyloid fibril formation, are anticipated to also be effective in the inhibition of alpha-synuclein fibril formation, particularly as observed in Parkinson's and Lewy body diseases. The agents and compounds disclosed herein will therefore also serve as therapeutics for Parkinson's and Lewy body disease, in addition to having efficacy as a therapeutic for Alzheimer's disease and other amyloid disorders.

Use of Catechins, Bioflavanoids, Flavanols, Flavandiols, Flavanoids, Tannins (or Derivatives of any of these) from Green Tea, Green Tea Leaves, Green Tea Extracts or Green Tea Derivatives, or from other Natural or Synthetic Sources

Green tea, and other natural sources such as black tea and wine, are known to contain catechins, bioflavanoids, flavanols, flavandiols, flavanoids, tannins or derivatives thereof. See Sugita-Konishi et al, “Epigallocatechin gallate and gallocatechin gallate in green tea catechins inhibit extracellular release of Vero toxin from enterohemorrhagic E. coli”, Biochemica et Biophysica Acta, 1472, 42-50(1999), and Fernandez et al, “HPLC determination of catechins and caffeine in tea”, Analyst 125: 421-425 (2000), the texts of which are hereby incorporated by reference as if fully set forth. These constituents are now believed by us to play a significant role in the surprisingly beneficial effects of green tea in amyloidoses as discussed below.

In particular the catechins of the group consisting of catechin, epicatechin, gallocatechin gallate, epigallocatechin gallate, epigallocatechin, and/or epicatechin gallate or a derivative of one of the above group, are now believed by us to be effective, either alone or in combination with other amyloid inhibitory ingredients such any plant matter from the group of plants consisting of, and commonly known as, Cat's claw, ginkgo biloba, rosemary, gotu kola, bacopin, and ginseng, to achieve any or all of the beneficial effects described below.

General structures for representative catechins are presented below (it is expected that various R-group type substitutions, and other derivative structural modifications, not affecting the disclosed efficacy of these compounds may be made, as will be appreciated by those skilled in the art, without affecting the scope of the appended claims):

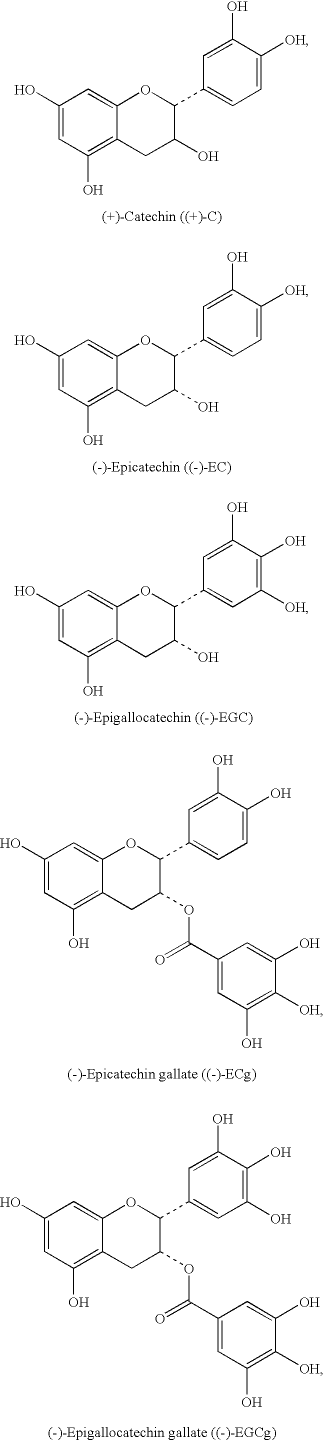

Use of Standardized Green Tea Leaf Extract to Inhibit Amyloidosis

The Examples illustrated below all serve well to establish that, at least in vitro, green tea, green tea leaves and extracts or derivatives thereof, have the ability to inhibit the formation of brain amyloid deposits that occur during normal aging and in a variety of brain disorders including Alzheimer's disease. In addition, it is known that patients who accumulate brain amyloid deposits eventually lose cognitive ability and memory function and sustain a marked reduction in mental clarity in general. Therefore it follows that inhibition of such brain amyloid deposits will at the least promote mental alertness in such patients.

The Examples also establish that again, at least in vitro, green tea, green tea leaves and extracts or derivatives thereof, have the ability to reduce, eliminate, prevent, inhibit, disrupt/dissolve, or disaggregate amyloid fibril or protein deposits, as well as amyloid fibril formation, or age associated amyloid fibril formation, and brain associated amyloid fibril formation. In addition, it is known that patients who accumulate amyloid fibril or protein deposits, brain associated fibril deposits or brain associated amyloid protein deposits, or who display symptoms of amyloid fibril formation and growth or age associated amyloid fibril formation and growth, brain associated amyloid fibril formation and growth, in general will eventually lose mental acuity, mental alertness, concentration, cognitive well being, or some measure of brain function or cognitive ability, mental performance or memory, or concentration and mental sharpness, or mental vitality, or mental clarity and alertness, short term memory, or some of the ability to learn and remember. It is also known that such patients are subject to age associated or related cognitive or memory decline, or will sustain a marked reduction in mental clarity. It follows then that inhibition, reduction, elimination, prevention, disruption, disassembly or disaggregation of such amyloid fibril or protein deposits, brain associated amyloid fibril deposits or brain associated amyloid protein deposits, or amyloid fibril formation and growth, or age associated amyloid fibril formation and growth, will improve mental acuity, promote mental alertness, provide nutritional support for age related cognitive or memory decline, promote cognitive well being, support brain function, improve cognitive ability, mental performance or memory, promote concentration and mental sharpness, improve mental vitality, promote greater mental clarity and alertness, improve short term memory, reduce or reverse age associated cognitive or memory decline, support normal brain function, enhance learning or memory; improve concentration, enhance mental performance, reduce mental decline, reduce likelihood of age related brain disorders, and maintain good brain health, in such patients.

The Examples further suggest that green tea, green tea leaves and extracts or derivatives thereof, should have the ability to reduce, eliminate, prevent, inhibit, disrupt, dissolve, disassemble, disaggregate amyloid fibril or protein deposits, pancreas associated amyloid fibril or protein deposits, as well as amyloid fibril formation and growth, and pancreas associated amyloid fibril formation and growth. In addition, it is known that patients who accumulate amyloid fibril or protein deposits, pancreas associated amyloid fibril or protein deposits, or who display symptoms of amyloid fibril formation and growth, pancreas associated amyloid fibril formation and growth, in general lose healthy pancreatic function or sustain a reduction in normal insulin function, leading to loss or reduction of pancreatic function. It there follows that inhibition, reduction, elimination, prevention, disruption, disassembly, dissolution or disaggregation of such amyloid fibril or protein deposits, pancreas associated amyloid fibril or protein deposits, or amyloid fibril formation and growth, pancreas associated amyloid fibril formation and growth, will support healthy pancreatic function and promote pancreatic function by helping to promote normal insulin function in such patients.

Recent studies, published after the priority date of this application, support these experimental results. See Hasegawa, “Preventive effect of Japanese green tea against cognitive impairment in the elderly”, posters 42 and 755, proceedings of World Alzheimer Congress 2000, in Neurobiology of Aging, 21:18 (2000), reporting statistically significant relationship between increased drinking of green tea and higher cognitive levels.

### EXAMPLES

The following examples are put forth so as to provide those with ordinary skill in the art with the disclosure and description and use of commercially available green tea extract which surprisingly are shown to cause an inhibition, disassembly/disruption and/or disaggregation of Alzheimer's disease Aβ-containing fibrils. However, it should not be construed that the invention is limited to these specific examples.

### Example 1

Standardized Green Tea Leaf Extract is a Potent Inhibitor of Alzheimer's Aβ (1-40) Amyloid Fibril Formation

A previously described method of measuring amyloid fibril formation utilizing Thioflavin T fluorometry (H Naiki et al, Lab. Invest. 65:104-110, 1991; H Levine III, Protein Sci. 2:404-410, 1993; H Levine III, Amyloid: Int. J. Exp. Clin. Invest. 2:1-6, 1995; H Naiki and K. Nakakuki, Lab. Invest. 74:374-383, 1996) was employed initially to identify whether standardized green tea leaf extract was capable of inhibiting Alzheimer's Aβ amyloid fibril formation. Using this sensitive assay, any decreases or increases in fluorescence was previously shown to correlate with a decrease or increase in the amount of amyloid fibrils (H Naiki et al, Lab. Invest. 65:104-110, 1991; H Levine III, Protein Sci. 2:404-410, 1993; H Levine III, Amyloid: Int. J. Exp. Clin. Invest. 2:1-6, 1995; H Naiki and K. Nakakuki, Lab. Invest. 74:374-383, 1996), allowing one to determine the effects of potential inhibitors and/or enhancers of amyloid fibril formation.

In a first study, the effects of standardized green tea extract as a potent Alzheimer's disease amyloid inhibitory agent on Alzheimer's Aβ (1-40) fibril formation was assessed by Thioflavin T fluorometry. Thioflavin T is known to bind to fibrillar amyloid proteins, and an increase in fluorescence correlates with an increase in amyloid fibril formation, whereas a decrease in fluorescence correlates with a decrease in amyloid fibril formation. The Alzheimer's Aβ protein (1-40) when incubated at 37° C. tends to spontaneously form amyloid fibrils which increase in quantity over time. In this study, we tested for standardized green tea extract to inhibit the Alzheimer's amyloid Aβ protein from forming fibrils over a 1 week period. For this study, 25 μM of Aβ (1-40)(Bachem Inc., Torrance, Calif., USA; Lot #WM365) was incubated in microcentrifuge tubes at 37° C. for 1 week (in triplicate), either alone, or in the presence of 10 μg/ml, 50 μg/ml or 100 μg/ml of standardized green tea extract in 150 mM Tris HCl, 10 mM NaCl, pH 7.0 (TBS). For this study, the powder within one gelatin capsule of standardized green tea extract obtained from a commercial source (Nature's Resource, Mission Hills, Calif.) was extracted in 1 ml of distilled water and pelleted using a microcentrifuge (for 10 minutes at 2,500×g). The supernatant was then taken and lyophilized. A 1 mg/ml working solution for use in the in vitro assays described below was then made using distilled water. The commercial green tea leaf extracts are usually standardized to 50% polyphenols.

To assess the effects of standardized green tea extract on Aβ (1-40) fibril formation, 50 μl aliquots were taken from each tube for analysis at 1 hr, 1 day, 3 days, and 1 week. For each determination described above, following each incubation period, 50 μl of Aβ+/−standardized green tea extract were added to 1.2 ml of 100 μM Thioflavin T (Sigma Chemical Co., St. Louis, Mo.) in 50 mM NaPO₄ (pH 6.0). Studies indicated that increasing concentrations of fibrillar Aβ gave a proportional increase in fluorescence in the presence of 100 μM Thioflavin T, ruling out the presence of any disproportionate inner filter effects in these studies. Fluorescence emission at 482 nm was measured on a Turner instrument-model 450 fluorometer at an excitation wavelength of 450 nm. For each determination, the fluorometer was calibrated by zeroing in the presence of the Thioflavin T reagent alone, and by setting the 50 ng/ml riboflavin (Sigma Chemical Co., St. Louis, Mo.) in the Thioflavin T reagent to 1800 fluorescence units. All fluorescence determinations were based on these references and any fluorescence given off by any of the compounds in the presence of the Thioflavin T reagent was always subtracted from all pertinent readings.

For all fibrillogenesis studies utilizing Thioflavin T fluorometry, as disclosed herein, comparisons of amyloid protein in the presence or absence of standardized green tea extract were based on paired Student's t tests with data shown as mean+/−standard deviation. Significance was reported at the 95% (p<0.05),99% (p<0.01) and 99.5% (p<0.005) confidence levels.

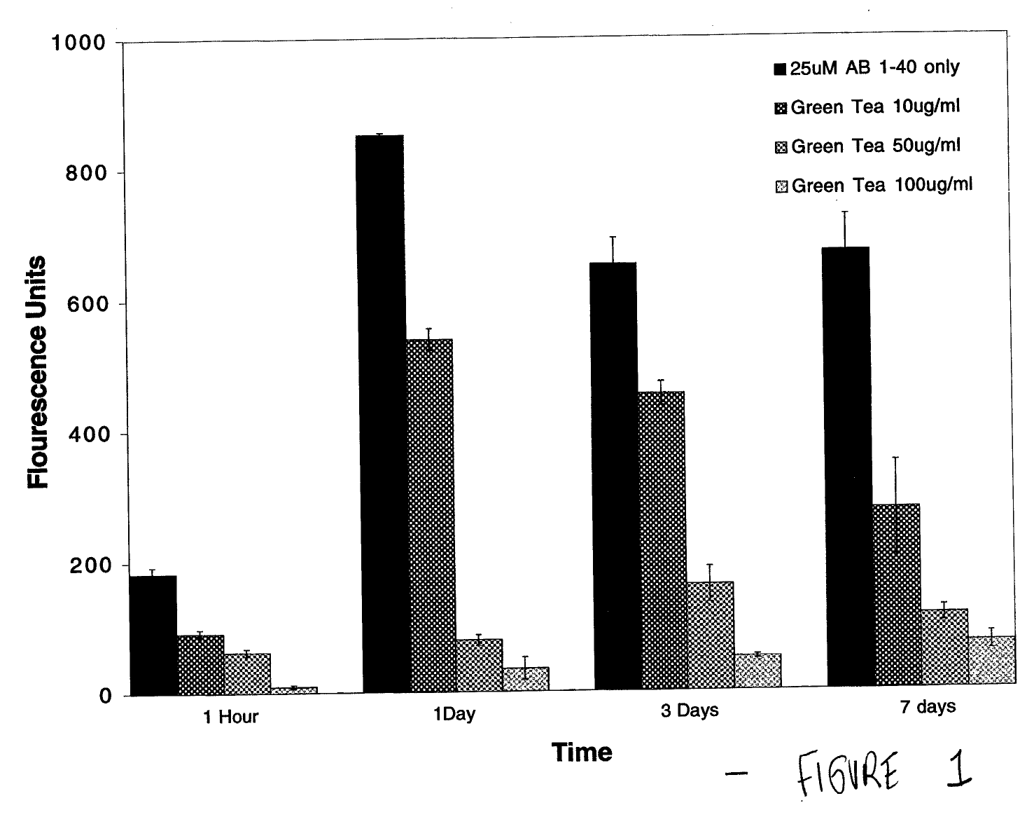

As shown in FIG. 1, the effects standardized green tea extract on Alzheimer's Aβ (1-40) amyloid fibril formation was evaluated over a 1 -week incubation period. Freshly suspended Aβ (1-40) alone, following a 1-hour incubation at 37° C., demonstrated an initial fluorescence of 183+/−10 fluorescence units. During the 1 -week incubation period, there was a gradual increase in the fluorescence of Aβ (1-40) alone, increasing approximately 4-fold from 1 hour to 1 day, with a peak fluorescence of 852+/−3 fluorescence units observed at 1 day (FIG. 1), consistent with previous studies (Castillo et al, J. Neurochem. 69:2452-2465, 1997). Standardized green tea extract significantly inhibited Aβ (1-40) amyloid fibril formation in a dose-dependent manner as early as 1 hour of incubation (FIG. 1). Significant inhibition (p<0.005) by standardized green tea leaf extract on Aβ 1-40 amyloid fibril formation was observed at all time points including 1 hour, 1 day, 3 days and 1 week (FIG. 1). At 1 hour, 10μg/ml, 50 μg/ml and 100 μg/ml of standardized green tea leaf extract significantly (p<0.005) inhibited Aβ 1-40 fibril formation by 50.3+/−3.3%, 66.1+/−3.3%, and 95.1+/−1.6%, respectively. At 1 day, 10 μg/ml, 50 μg/ml and 100 μg/ml of standardized green tea leaf extract significantly (p<0.005) inhibited Aβ 1-40 fibril formation by 36.7+/−2.0%, 90.7+/−1.0%, and 95.9+/−2.0%, respectively. By 1 week, 10 μg/ml, 50 μg/ml and 100 μg/ml of standardized green tea leaf extract significantly (p<0.005) inhibited Aβ 1-40 fibril formation by 58.9+/−10.7%, 83.0+/−1.8%, and 89.3+/−2.1%, respectively. This initial data indicated that standardized green tea extract and at least one of its catechin, bioflavanoid, flavanol, flavandiol, flavanoid or tannin active constituents was a potent inhibitor of Alzheimer's amyloid fibril formation.

### Example 2

Disassembly/Disruption of Alzheimer's Disease Aβ 1-42 Amyloid Fibrils by Standardized Green Tea Leaf Extract

In the next study, standardized green tea leaf extract was tested for its ability to cause a disassembly/disruption of pre-formed Alzheimer's disease amyloid fibrils containing Aβ 1-42. This type of activity would be important for any potential anti-amyloid compound which can be used in patients who already have substantial amyloid deposition in organs and/or tissues. For example, Alzheimer's disease patients in mid-to-late stage disease have abundant Aβ-containing amyloid deposits in their brains as part of both neuritic plaques and cerebrovascular amyloid deposits. A compound capable of causing disassembly/disruption of pre-existing amyloid deposits would be advantageous for use in these patients who are at latter stages of the disease process.

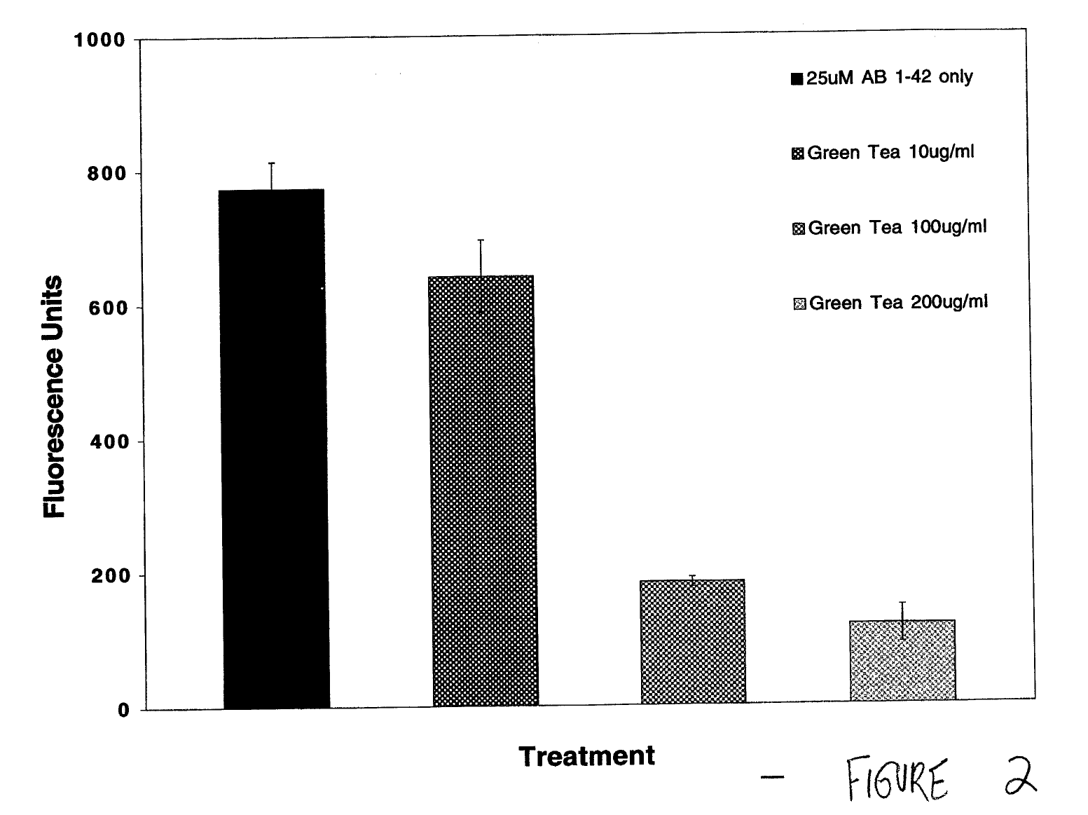

For this study, 1 mg of Aβ 1-42 (Bachem Inc., Torrance, Calif., USA; Lot #516817) was dissolved in 1.0 ml of double distilled water (1 mg/ml solution). 25 μM of Aβ 1-42 was then incubated overnight (˜18 hours) at 37° C., in the absence or presence of 10 μg/ml, 100 μg/ml or 200 μg/ml of standardized green tea leaf extract in the presence of 150 mM Tris HCl, 10 mM NaCl (pH 7.0) with 0.02% sodium azide. In these studies (see results described below and in FIG. 2), the Aβ 1-42:green tea extract weight ratio was 1:0.1, 1:1 and 1:2, respectively.

For this study, the powder within one gelatin capsule of standardized green tea extract obtained from a commercial source (Nature's Resource, Mission Hills, Calif.) was extracted in 1 ml of distilled water and pelleted using a microcentrifuge (for 10 mins at 2,500×g). The supernatant was then taken and lyophilized. A 1 mg/ml working solution for use in the in vitro assays described below was then made using distilled water. The commercial green tea leaf extracts are usually standardized to 50% polyphenols.

A previously described method of measuring amyloid fibril formation utilizing Thioflavin T fluorometry (H Naiki et al, Lab. Invest. 65:104-110, 1991; H Levine III, Protein Sci. 2:404-410, 1993; H Levine III, Amyloid: Int. J. Exp. Clin. Invest. 2:1-6, 1995; H Naiki and K. Nakakuki, Lab. Invest. 74:374-383, 1996) was employed to assess whether standardized green tea leaf extract is capable of causing a disassembly/disruption of Alzheimer's Aβ 1-42 amyloid fibrils. Thioflavin T is known to bind to fibrillar amyloid proteins, and an increase in fluorescence correlates with an increase in amyloid fibril formation, whereas a decrease in fluorescence correlates with a decrease in amyloid fibrils due to disassembly and/or disruption. The Alzheimer's Aβ protein (1-42) when placed in solution, such as distilled water, tends to spontaneously form amyloid fibrils. Using this sensitive assay, any decreases or increases in fluorescence was previously shown to correlate with a decrease or increase in the amount of amyloid fibrils (H Naiki et al, Lab. Invest. 65:104-110, 1991; H Levine III, Protein Sci. 2:404-410, 1993; H Levine III, Amyloid: Int. J. Exp. Clin. Invest. 2:1-6, 1995; H Naiki and K. Nakakuki, Lab. Invest. 74:374-383, 1996), allowing one to identify, and quantitate the extent of potential inhibitors and/or enhancers of Alzheimer's Aβ 1-42 amyloid fibrils.

To assess the effects of standardized green tea extract on potential disassembly/ disruption of preformed Aβ 1-42 fibrils, 50 μl of Aβ 1-42+/−standardized green tea leaf extract at various concentrations (described above) were added to 1.2 ml of 100 μM Thioflavin T (Sigma Chemical Co., St. Louis, Mo.) in 50 mM NaPO4 (pH 6.0) for fluorometry readings (as described in Example 1).

As shown in FIG. 2, increasing amounts of standardized green tea extract caused a dose-dependent disassembly/disruption of pre-formed Alzheimer's Aβ 1-42 fibrils. Aβ 1-42 alone demonstrated a mean fluorescence of 780+/−50 fluorescence units (FIG. 2). Standardized green tea extract at 10 μg/ml significantly (p<0.05) caused a disassembly/disruption of Aβ 1-42 fibrils by 17+7%. On the other hand, 100 μg/ml and 200 μg/ml significantly (p<0.005) caused a disassembly/disruption of Aβ 1-42 fibrils by 76+/−1.0% and 85+/−4.0%, respectively. This study demonstrated that standardized green tea extract and at least one of its catechin, bioflavanoid, flavanol, flavandiol, flavanoid or tannin active constituents caused disassembly/disruption of pre-formed Aβ 1-42 amyloid fibrils and was effective in a dose-dependent manner.

### Example 3

Disaggregation of Alzheimer's Disease Aβ 1-42 Fibrils by Standardized Green Tea Leaf Extract

In the next study a Congo red-Aβ 3 spectrophotometric assay (Klunk et al, Anal. Biochem. 266:66-76, 1999) was modified to determine the effectiveness of standardized green tea leaf extract on disaggregation of Alzheimer's Aβ 1-42 amyloid fibrils. For this assay, 25 μM of Aβ 1-42 (Bachem Inc., Torrance Calif., Lot #516817) was incubated in triplicate for 4 days in distilled water at 37° C. in the absence or presence of 400 μg/ml of standardized green tea extract (obtained from two commercial sources) in Tris-buffered saline (TBS)(100 mM Tris; 50 mM NaCl; pH 7.0, with 0.02% sodium azide). The Aβ: green tea extract weight ratio was 1:4. Source 1 of the standardized green tea extract used in this study was from Sundown Herbals (manufactured and distributed for Sundown Vitamins, Boca Raton, Fla. ), whereas source 2 of the standardized green tea extract used in this study was form Nature's Resource (Mission Hills, Calif.). The green tea used in this study were extracted in distilled water as described in Examples 1 and 2.

Following incubation of Aβ 1-42 in the presence or absence of standardized green tea extracts (as described above)(standardized green tea extracts from the 2 sources are referred to as test compounds), 50 μl of 360 μM of Congo red (Sigma Chemical Co. St. Louis, Mo., USA) in distilled water was then added to 250 μl of each incubation mixture, giving a final Aβ:Congo red molar ratio of 1:3. After 10 minutes, the absorbance at 405 nm (reference wavelength to account for the absorbance of Congo red alone at 540 nm) and 540 nm (sample absorbance where “sample” refers to Aβ alone, test compound alone, or Aβ+test compound, all in the presence of Congo red) was determined using a Biorad Model 550 ELISA Plate Reader (Biorad, Hercules, Calif., USA). The absorbance at wavelength 405 nm was automatically subtracted by the ELISA plate reader from the absorbance at wavelength 540 nm (difference is referred to as Δ absorbance)(Klunk et al, Anal. Biochem. 266:66-76, 1999). Therefore, the Δ absorbance reading at 540 nm was proportional to the amount of aggregated Aβ left in solution (Klunk et al, Anal. Biochem. 266:66-76, 1999).

For all experiments involving test compounds, the Δ absorbance reading at 540 nm of the test compound alone (in the absence of Aβ), was always subtracted from the corresponding Δ absorbance reading at 540 nm of the test compound in the presence of Aβ.

Using this modification of the method of Klunk et al (Anal. Biochem. 266:66-76, 1999), the use of a greater final concentration of Congo red (i.e. 60 μM instead of 14 μM)(Anal. Biochem. 266:66-76, 1999), in the presence of fibrillar Aβ gave an overall absorbance at 540 nm that was always below 1.0 Absorbance Unit (AU), and well within the linear absorbance range.

Standardized green tea leaf extracts from two commercial sources were tested using the above described Congo red-Aβ spectrophotometric assay to determine their effectiveness on disaggregation of Aβ 1-42 Alzheimer's amyloid fibrils.

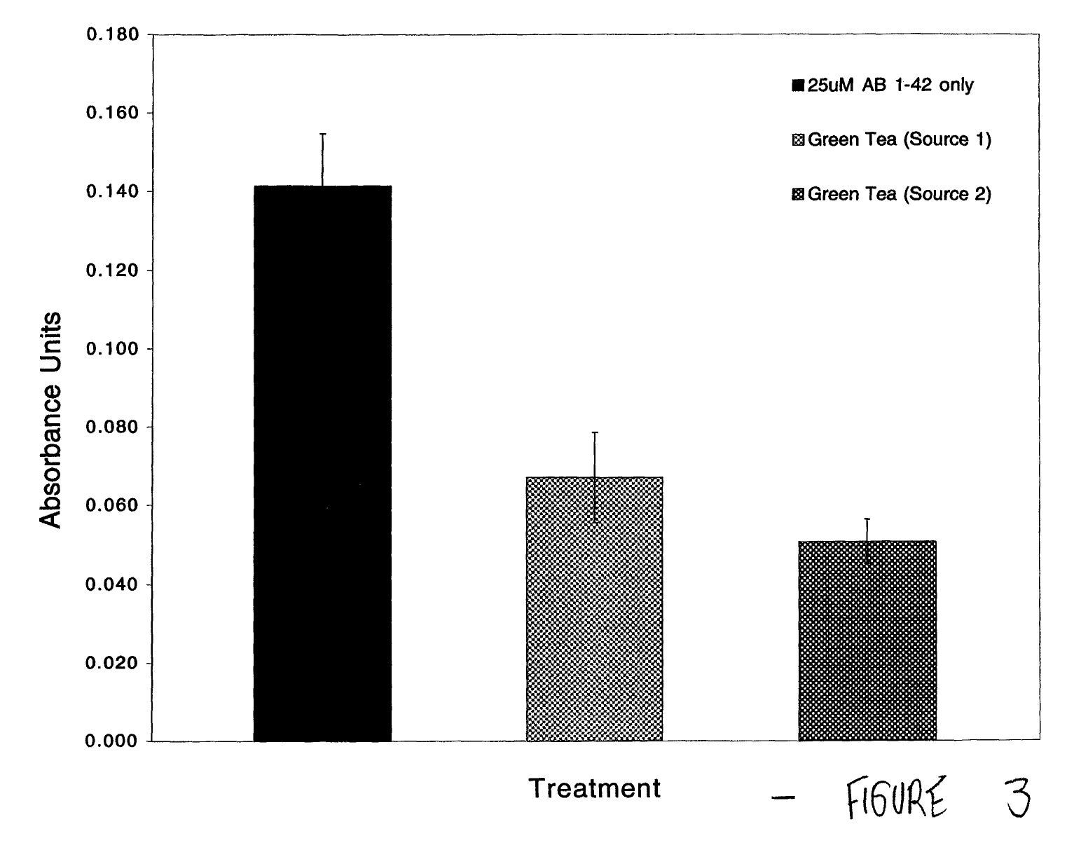

As shown in FIG. 3, both commercially available standardized green tea extracts caused a disaggregation of pre-aggregated Aβ 1-42 amyloid fibrils as determined using the Congo red spectrophotometric assay described above. Aβ 1-40 alone demonstrated a mean Δ absorbance of 0.141+/−0.013 AU (FIG. 3). Standardized green tea extract from source 1 (Sundown Herbals) caused a significant (p<0.005) 52.5+/−8.5% disaggregation of 25 μM Aβ 1-42 fibrils when incubated at a Aβ 1-42:green tea extract weight ratio of 1:4 (FIG. 3). On the other hand, standardized green tea extract from source 2 (Nature's Resource) caused a significant (p<0.005) 63.8+/−4.2% disaggregation of 25 μM Aβ 1-42 fibrils when incubated at a Aβ 1-42:green tea extract weight ratio of 1:4 (FIG. 3). Thus, independent of source, standardized green tea leaf extract and at least one of its catechin, bioflavanoid, flavanol, flavandiol, flavanoid or tannin active constituents was a potent disaggregator of Alzheimer's Aβ 1-42 amyloid fibrils.

### Industrial Applicability

The invention finds worldwide utility in that it provides therapeutic relief and diagnostic assistance in treating and preventing Alzheimer's disease and other amyloidoses by use of a readily occurring and relatively inexpensive plant ingredient.

In compliance with the statute, the invention has been described in language more or less specific as to structural features. It is to be understood, however, that the invention is not limited to the specific features shown, since the means and construction shown comprise preferred forms of putting the invention into effect. The invention is, therefore, claimed in any of its forms or modifications within the legitimate and valid scope of the appended claims, appropriately interpreted in accordance with the doctrine of equivalents.

We claim:

1. A method of treatment, prevention, or management of an amyloidosis in a mammalian subject susceptible to, or afflicted by, the amyloidosis, the method comprising the step of administering to the subject a therapeutic amount of plant matter from a source of green tea, green tea leaves, standardized green tea extract, or green tea derivative.

2. The method of claim 1, wherein the amyloidosis is selected from the group of amyloidoses consisting of Alzheimer's disease, type II diabetes, Down's syndrome, hereditary cerebral hemorrhage with amyloidosis of the Dutch type, the amyloidosis associated with chronic inflammation, various forms of malignancy and familial Mediterranean fever, the amyloidosis associated with multiple myeloma and other B-cell dyscrasias, the amyloidosis associated with type II diabetes, the amyloidosis associated with the prion diseases including Creutzfeldt-Jakob disease, Gerstmann-Straussler syndrome, kuru and animal scrapie, the amyloidosis associated with long-term hemodialysis and carpal tunnel syndrome, the amyloidosis associated with senile cardiac amyloid and familial amyloidosis polyneuropathy, and the amyloidosis associated with endocrine tumors such as medullary carcinoma of the thyroid.

3. The method of claim 2, wherein the amyloidosis is Alzheimer's disease.

4. A method for the treatment, inhibition, prevention or management of amyloid formation, deposition, accumulation, aggregation and/or persistence in Alzheimer's disease, type II diabetes and other amyloidoses in a mammalian subject, the method comprising the step of administering to the subject a therapeutic amount of a substance selected from the group of substances consisting of green tea, green tea leaves, standardized green tea extract, green tea derivative, catechins, bioflavanoids, flavanols, flavandiols, flavanoids, tannins or derivatives thereof.

5. The method of claim 4, wherein the substance is a catechin selected from the group of catechins consisting of catechin, epicatechin, gallocatechin gallate, epigallocatechin gallate, epigallocatechin, and epicatechin gallate, or a derivative of one of the above group.

6. A method for the treatment, prevention or management of an amyloidosis in a mammalian subject susceptible to the amyloidosis, the method comprising the step of administering to the subject a therapeutic amount of a substance selected from the group of substances consisting of catechins, bioflavanoids, flavanols, flavandiols, flavanoids, tannins or derivatives thereof.

7. The method of claim 6, wherein the substance is a catechin selected from the group of catechins consisting of catechin, epicatechin, gallocatechin gallate, epigallocatechin gallate, epigallocatechin, and epicatechin gallate, or a derivative of one of the above group.

8. The method of claim 6, wherein the amyloidosis is selected from the group of amyloidoses consisting of Alzheimer's disease, type II diabetes, Down's syndrome, hereditary cerebral hemorrhage with amyloidosis of the Dutch type, the amyloidosis associated with chronic inflammation, various forms of malignancy and Familial Mediterranean Fever, the amyloidosis associated with multiple myeloma and other B-cell dyscrasias, the amyloidosis associated with type II diabetes, the amyloidosis associated with the prion diseases including Creutzfeldt-Jakob disease, Gerstmann-Straussler syndrome, kuru and animal scrapie, the amyloidosis associated with long-term hemodialysis and carpal tunnel syndrome, the amyloidosis associated with senile cardiac amyloid and familial amyloidosis polyneuropathy, and the amyloidosis associated with endocrine tumors such as medullary carcinoma of the thyroid.

9. The method of claim 8, wherein the amyloidosis is Alzheimer's disease.

10. The method of claim 1 further comprising, in the step of administering plant matter, additionally administering a therapeutic quantity of one or more plant materials selected from the group of plants consisting of, and commonly known as, Cat's claw, ginkgo biloba, rosemary, gotu kola, bacopin, and ginseng.

11. A method for the treatment, inhibition, prevention or management of α-synuclein fibril formation, deposition, accumulation, aggregation and/or persistence in Parkinson's disease or Lewy body disease in a mammalian subject, the method comprising the step of administering to the subject a therapeutic amount of a substance selected from the group of substances consisting of green tea, green tea leaves, standardized green tea extract, green tea derivative, catechins, bioflavanoids, flavanols, flavandiols, flavanoids, tannins or derivatives thereof.

12. The method of claim 11, wherein the substance is a catechin selected from the group of catechins consisting of catechin, epicatechin, gallocatechin gallate, epigallocatechin gallate, epigallocatechin, and epicatechin gallate, or a derivative of one of the above group.

13. A method for promoting mental alertness in a patient, the method comprising the step of administering to the patient a therapeutically effective amount of plant matter from a plant of the family Theaceae.

14. The method of claim 13 wherein the plant matter comprises matter from a plant of the genus Camellia, species sinensis.

15. The method of claim 13 wherein the method is also for inhibiting the formation of brain amyloid deposits.

16. A method for promoting, maintaining or enhancing in a patient one or more of the mental or cognitive qualities selected from the group of mental or cognitive qualities consisting of mental acuity, mental alertness, cognitive well being, normal brain function, cognitive ability, mental performance, memory, concentration, mental sharpness, mental clarity, short term memory, normal brain function, and learning, the method comprising the step of administering to the patient a therapeutically effective amount of plant matter from a plant of the genus Camellia, species sinensis.

17. A method for providing, supporting or improving in a patient one or more of the mental or cognitive qualities selected from the group of mental or cognitive qualities consisting of normal brain function, cognitive ability, and concentration, the method comprising the step of administering to the patient a therapeutically effective amount of plant matter from a plant of the genus Camellia, species sinensis.

18. A method for reducing in a patient one or more of the mental or cognitive effects selected from the group of mental or cognitive effects consisting of, age associated cognitive or memory decline, mental decline, and likelihood of age related brain or cognitive disorders, the method comprising the step of administering to the patient a therapeutically effective amount of plant matter from a plant of the genus Camellia, species sinensis.

19. A method for reducing, disrupting, dissolving, inhibiting, eliminating or preventing in a patient one or more conditions involving the brain selected from the group of conditions involving the brain consisting of amyloid fibril deposits, amyloid protein deposits, brain associated amyloid fibril deposits, brain associated amyloid protein deposits, amyloid fibril formation and growth, age associated amyloid fibril formation and growth, brain associated amyloid fibril formation and growth, the method comprising the step of administering to the patient a therapeutically effective amount of plant matter from a plant of the genus Camellia, species sinensis.

20. A method for promoting or supporting healthy pancreatic function in a patient, by helping to promote normal insulin function, the method comprising the step of administering to the patient a therapeutically effective amount of plant matter from a plant of the genus Camellia, species sinensis.

21. A method for reducing, disrupting, dissolving, inhibiting, eliminating or preventing in a patient one or more conditions involving the pancreas selected from the group of conditions involving the pancreas consisting of amyloid fibril deposits, amyloid protein deposits, pancreas associated amyloid fibril deposits, pancreas associated amyloid protein deposits, amyloid fibril formation and growth, pancreas associated amyloid fibril formation and growth, the method comprising the step of administering to the patient a therapeutically effective amount of plant matter from a plant of the genus Camellia, species sinensis.
